(19) United States
(12) Patent Application Publication
(10) Pub. No.: US 2023/0154306 A1
(43) Pub. Date: May 18, 2023

(54) ARTIFICIAL INTELLIGENCE BASED SMART HAND SANITIZATION SYSTEM FOR ELDERLY

(71) Applicants: Deepak Srivastava, Dehradun (IN); Dilip Kumar J. Saini, Dehradun (IN); Neeraj Rathore, Dehradun (IN); Shachi Mall, Gorakhpur (IN); Ashutosh Bhatt, Dehradun (IN); Suryabhan Pratap Singh, Gorakhpur (IN); Rajkumar Jain, Indore (IN); Pranjal Maurya, Gorakhpur (IN); Shailesh Kumar, Noida (IN); Devendra Prasad, Dehradun (IN); Seema Garg, Noida (IN)

(72) Inventors: Deepak Srivastava, Dehradun (IN); Dilip Kumar J. Saini, Dehradun (IN); Neeraj Rathore, Dehradun (IN); Shachi Mall, Gorakhpur (IN); Ashutosh Bhatt, Dehradun (IN); Suryabhan Pratap Singh, Gorakhpur (IN); Rajkumar Jain, Indore (IN); Pranjal Maurya, Gorakhpur (IN); Shailesh Kumar, Noida (IN); Devendra Prasad, Dehradun (IN); Seema Garg, Noida (IN)

(21) Appl. No.: 17/691,035

(22) Filed: Mar. 9, 2022

(30) Foreign Application Priority Data
Nov. 12, 2021 (IN) 202111051874

(51) Int. Cl. G08B 21/24; G08B 21/04; G08B 7/06; A61L 2/00; A61L 2/24

(52) U.S. Cl. CPC G08B 21/245; G08B 21/04; G08B 7/06; A61L 2/0088; A61L 2/24; A61L 2202/14; A61L 2202/15

(57) ABSTRACT

The present invention relates to an artificial intelligence based smart hand sanitization system (100) for elderly is provided. The system (100) comprises an inlet pipe (102), a liquid chamber (104), a washing model (110), a collection chamber (106), and an outlet pipe (108). The washing model (110) comprises a plurality of sensors (112), a first scanner unit (116), and a plurality of moveable nozzles (114), a second scanner unit (118), a central processing unit (120), and an alert generating unit (122). The smart portable system (100) for hand-wash has a plurality of moveable nozzles (114) to wash the hands of the user without wetting the accessories and cloth of the user. The present invention is a detachable smart portable system (100) for hand wash that can detach from the wall and be re-fixed on the wall according to the user requirement.

This application claims priority to Indian Patent Application number 202111051874 filed on Nov. 12, 2021.

### TECHNICAL FIELD

The present invention relates to a system for the washing of hands, and more particularly, to an artificial intelligence based smart hand sanitization system for elderly.

### BACKGROUND

According to the healthcare department, keeping hands clean is one of the important steps to safe our-self from getting sick and spreading germs to others. Many diseases and conditions are spread by not properly washing hands with soap and clean, running water. The conventional system suffers from many problems like bulkiness, consuming more water, consuming more electrical energy, and consuming more soap cause wastage of soap. Also, the conventional hand-washer system wets the accessories and cloth of the user during washing hands. In old age, the hand of the user shakes due to which they are unable to wash their hand properly and find it difficult to hold their hands steady under the stream of water. The conventional automated hand washes system also fails to provide proper pressure to clean dirt and germs like grease or to clean the dirt and germs between the finger spaces.

Therefore, there remains a need in the art for an artificial intelligence based smart hand sanitization system for elderly that does not suffer from the above-mentioned deficiencies or at least provides a viable and effective solution.

### OBJECTIVE OF THE INVENTION

Some of the objects of the present disclosure, which at least one embodiment herein satisfies, are as follows.

It is an object of the present disclosure to ameliorate one or more problems of the prior art or to at least provide a useful alternative.

An object of the present disclosure is to provide an artificial intelligence-based smart hand sanitization system for elderly.

An object of the present disclosure is to provide a smart portable system for washing hands.

An object of the present disclosure is to provide a detachable smart portable system for hand wash.

An object of the present disclosure is to provide a smart portable system for hand-wash having a scanner to scan the accessories, cloth of the user, kind of dirt, and wound present on the hand of the user.

An object of the present disclosure is to provide a smart portable system for hand-wash having a moveable nozzle to wash the hand of the user without wetting the accessories and cloth wear of the user.

An object of the present disclosure is to provide a smart portable system for hand wash that facilitates dispensing of liquid in the desired intensity that can be varied automatically by regulating the pressure at which it is dispensed.

An object of the present disclosure is to provide a detachable smart portable system for a hand wash that can be detached from the wall and re-fixed on the wall according to the user requirement/or can be attached to a trolley with wheels for comfortable shifting from place to place.

An object of the present disclosure is to provide a smart portable system for the hand wash, which is compact requires less installation space, is easy to handle, and moves from one place to another place.

An object of the present disclosure is to provide a detachable smart portable system for hand wash which reduces the consumption of energy, wastage of water, and wastage of soap/or detergent.

Another object of the present disclosure is to provide a smart portable system for hand wash to analyze the condition of the containers, condition of the hand after wash, and generate an alert to correct the issue.

### SUMMARY

The following presents a simplified summary of the invention to provide a basic understanding of some aspects of the invention. This summary is not an extensive overview of the present invention. It is not intended to identify the key/critical elements of the invention or to delineate the scope of the invention. Its sole purpose is to present some concept of the invention in a simplified form as a prelude to a more detailed description of the invention presented later.

An embodiment of the present invention provides an artificial intelligence based smart hand sanitization system for elderly. The system comprises an inlet pipe, a liquid chamber, a washing model, a collection chamber, and an outlet pipe. The inlet pipe is configured to fill liquids inside the system. The inlet pipe is attached to the liquid chamber. The liquid chamber comprises one or more separating walls. The one or more separation walls are configured to separately carry the received liquids. The liquid chamber is operatively connected with the washing model. The washing model has one or more hand receiving holes. The hand receiving holes is configured to provide entering gates for the user hands in the system for washing. The washing model comprises a plurality of sensors, a first scanner unit, a plurality of moveable nozzles, a second scanner unit, a central processing unit, and an alert generating unit. The one or more hand receiving holes provide housing to the first scanner unit. The plurality of sensors is configured to detect conditional information about the system. The first scanner unit is configured to scan the hands of the user and generate the first scan information. The plurality of moveable nozzles operationally is connected with the first scanner unit. The second scanner unit is operatively connected to the plurality of moveable nozzles. The second scanner unit is configured to scan dirt and germs present in the hand of the user after wash and generate second scan information. The alert generating unit is operationally connected with the plurality of sensors, second scanner unit, liquid chamber and inlet pipe. The alert generating unit is configured to generate the alert. The central processing unit is operationally connected with the first scanner unit, the plurality of moveable nozzles, and the second scanner unit. The central processing unit is configured to receive the scanned information of the user's hands by the first scanner unit; generate moveable nozzle operating signal according to the received scanned information and send the operating signal to the plurality of moveable nozzles; operate the plurality of moveable nozzles, discharge the soap and water to wash the hands of the user according to the generated operating signal; receive the second scanned information by the second scanner unit and the conditional information about the system by the plurality of sensors; generate and send the alert signal to the alert generating unit to generate an alert. The collection chamber is operatively connected with the washing module. The collection chamber is configured to collect the dirty water produced after hand washing. The outlet pipe is attached to the collection chamber. The outlet pipe is configured to drain out dirty water from the system.

In accordance with an embodiment of the present invention, the liquid may be selected from a group comprising liquid soap, water, liquid antiseptic, sanitizer or a combination thereof.

In accordance with an embodiment of the present invention, the conditional information about the system may be filling level of liquid, leakage of water from collection chamber and liquid chamber, presence of user nearby system.

In accordance with an embodiment of the present invention, the plurality of sensors is selected from a group comprising a water leakage sensor (ultrasonic sensor), a proximity sensor, hydrostatic pressure level sensor, radar level sensor, ambient light sensor, ultrasonic proximity, IR sensor proximity sensor, capacitive proximity sensor, passive IR sensor.

In accordance with an embodiment of the present invention, the first scan information is a type of wound, type of accessories, shape, and size of accessories, type of dirt, and germs present on the hands of the user.

In accordance with an embodiment of the present invention, the second scan information is whether the hands of the user are properly clean or not, the presence of din and germs after wash, presence of din in between fingers after wash.

In accordance with an embodiment of the present invention, the first scanner unit is selected from a group comprising an infrared laser scanner, an optical scanner, IR optical scanner, UV scanner, or a combination thereof.

In accordance with an embodiment of the present invention, the second scanner unit is selected from a group comprising an infrared laser scanner, an optical scanner, IR optical scanner, UV scanner, or a combination thereof.

In accordance with an embodiment of the present invention, the alert generating unit is selected from a group comprising a sound generator, flashing light, short message service, image-producing unit, or combination thereof.

In accordance with an embodiment of the present invention, the alert signal is about the issues related to the leakage of water from collection chamber and liquid chamber, condition of filling level of liquid, not proper wash /or cleaning of hands, not proper working of the first scanner and second scanner, movement of nozzle, leakage of water through the inlet pipe and outlet pipe.

### BRIEF DESCRIPTION OF THE DRAWINGS

So that the manner in which the above-recited features of the present invention can be understood in detail, a more particular description of the invention, briefly summarized above, may have been referred to by embodiments, some of which are illustrated in the appended drawings. It is to be noted, however, that the appended drawings illustrate only typical embodiments of this invention and are therefore not to be considered limiting of its scope, for the invention may admit to other equally effective embodiments.

These and other features, benefits, and advantages of the present invention will become apparent by reference to the following text figure, with like reference numbers referring to like structures across the views, wherein

FIG. 1: Illustrates an artificial intelligence based smart hand sanitization system for elderly, in accordance with an embodiment of the present invention.

FIG. 2: Illustrates an implementation of the artificial intelligence based smart hand sanitization system for elderly, in accordance with an embodiment of the present invention.

### DETAILED DESCRIPTION

The following description is of exemplary embodiments only and is not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention.

While the present invention is described herein by way of example using embodiments and illustrative drawings, those skilled in the art will recognize that the invention is not limited to the embodiments of drawing or drawings described and are not intended to represent the scale of the various components. Further, some components that may form a part of the invention may not be illustrated in certain figures, for ease of illustration, and such omissions do not limit the embodiments outlined in any way. The drawings and detailed description thereto are not intended to limit the invention to the particular, form disclosed, but on the contrary, the invention is to cover all modifications, equivalents, and alternatives falling within the scope of the present invention as defined by the appended claim. As used throughout this description, the word “may” is used in a permissive sense (i.e. meaning having the potential to), rather than the mandatory sense, (i.e. meaning must). Further, the words “a” or “an” mean “at least one” and the word “plurality” means “one or more” unless otherwise mentioned. Furthermore, the terminology and phraseology used herein is solely used for descriptive purposes and should not be construed as limiting in scope. Language such as “including,” “comprising,” “having,” “containing,” or “involving,” and variations thereof, is intended to be broad and encompass the subject matter listed thereafter, equivalents, and additional subject matter not recited, and is not intended to exclude other additives, components, integers or steps. Likewise, the term “comprising” is considered synonymous with the terms “including” or “containing” for applicable legal purposes.

In accordance with an embodiment of the present invention, the list of essential part of the artificial intelligence based smart hand sanitization system (100) for elderly are mentioned below:

An artificial intelligence based smart hand sanitization system (100)

An inlet pipe (102)

A liquid chamber (104)

A collection chamber (106)

An outlet pipe (108)

A washing module (110)

A plurality of sensors (112)

A plurality of moveable nozzles (114)

A first scanner unit (116)

A second scanner unit (118)

A central processing unit (120)

An alert generating unit (122)

One or more hand receiving holes (124)

A covering module (126)

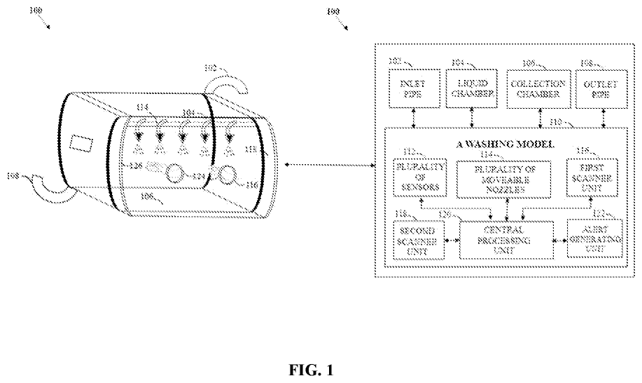

FIG. 1 illustrates an artificial intelligence based smart hand sanitization system (100) for elderly is provided in accordance with an embodiment of the present invention. The system (100) comprises an inlet pipe (102), a liquid chamber (104), a washing module (110), a collection chamber (106), and an outlet pipe (108). The inlet pipe (102) is configured to fill liquids inside the system (100). The inlet pipe (102) is operatively connected with the liquid chamber (104). The liquid chamber (104) is configured to store input liquids. The input liquids maybe, but are not limited to liquid soap, water, liquid antiseptic, sanitizer. The liquid chamber (104) comprises one or more separating walls (not shown in fig). The separating walls are configured to separately carry received liquids. The liquid chamber (104) is operationally connected with the washing model (110). The washing model (110) comprises a covering module (126). The covering module (126) is configured to avoid water spill out from the system (100) during hand wash and also provide more surface area for a comfortable hand wash. The covering module (126) provides housing to one or more hand receiving holes (124). The one or more hand receiving holes (124) are configured to provide an entry gate to the user's hands as shown in FIG. 1.

The washing model (110) further comprises a plurality of sensors (112). The plurality of sensors (112) is configured to detect conditional information about the system (100). The conditional information about the system maybe, but is not limited to filling level of liquid, leakage of water from the collection chamber (106) and liquid chamber (104), presence of user nearby system (100). The plurality of sensors (112) may be, but not limited to a water leakage sensor (ultrasonic sensor), a proximity sensor, hydrostatic pressure level sensor, radar level sensor, ambient light sensor, ultrasonic proximity, IR sensor proximity sensor, capacitive proximity sensor, passive IR sensor. Further, the washing module (110) comprises a first scanner unit (116). The one or more hand receiving holes (124) provide housing to the first scanner unit (116) as shown in FIG. 1. The first scanner unit (116) is configured to scan the hands of the user and generate first scan information. The first scan information maybe, but is not limited to a type of wound, type of accessories, shape, and size of accessories, type of dirt, and germs. The first scanner unit (116) may be, but not limited to an infrared laser scanner, an optical scanner, IR optical scanner, UV scanner.

Further, the washing model (110) further comprises a plurality of moveable nozzles (114). The plurality of moveable nozzles (114) is highly flexible in movement. The plurality of moveable nozzles (114) is operatively connected with the first scanner unit (116). The plurality of moveable nozzles (114) comprises a drive unit, a gear unit, a position management unit, external sensors, and a controller unit (not shown in FIG. 1). The drive unit is configured to provide proper movement to the plurality of moveable nozzles (114). The position management unit is configured to detect the distance between user hands and the plurality of moveable nozzles (114). The position management unit is further designed to detect the current position of the user's hands. The external sensors are configured to detect the position of the wound, accessories present on the user's hands. The drive unit, gear unit, position management unit, external sensors are operatively connected with the controller unit. The controller unit comprises a processing unit and a memory unit. The controller unit is configured to operate the plurality of moveable nozzles (114) according to the received information by the first scanner unit (116). The plurality of moveable nozzles (114) is further configured to spray liquids only on the required area of the user's hands. Additionally, the plurality of moveable nozzles (114) is configured to facilitate dispensing of liquids in the desired intensity that can be varied automatically by regulating the pressure at which it is dispensed.

Further, the washing model (110) comprises a second scanner unit (118). The second scanner unit (118) is operatively connected with the plurality of moveable nozzles (114). The functions of the second scanner unit (118) actuate when the function of the plurality of moveable nozzles (114) turns off. The second scanner unit (118) is configured to scan dirt and germs present on the hand of the user after wash and generate second scan information. The second scan information maybe but is not limited to whether the hands of the user are properly cleaned or not, the presence of dirt and germs after wash, presence of dirt in between fingers. The second scanner unit (118) maybe, but is not limited to an infrared laser scanner, an optical scanner, IR optical scanner, UV scanner.

Additionally, the washing model (110) comprises an alert generating unit (122). The alert generating unit (122) is operatively connected with the plurality of sensors (112), the second scanner unit (118), and the liquid chamber (104). The alert generating unit (122) is configured to receive an alert signal. The alert signal is about the issues related to the leakage of water from the collection chamber (106) and liquid chamber (104), condition of filling level of liquid, not proper wash /or cleaning of hands, not proper working of the first scanner and second scanner, leakage of water through the inlet pipe and outlet pipe. The alert generating unit (122) is configured to generate the alert on detecting the abnormal condition. The alert generating unit (122) maybe but is not limited to a sound generator, flashing light, short message service, image-producing unit.

Further, the washing model (110) comprises a central processing unit (120). The central processing unit (120) is operationally connected with the plurality of sensors (112), the first scanner unit (116), the plurality of moveable nozzles (114), the second scanner unit (118), and the alert generating unit (122). The central processing unit (120) is configured to control the function of the first scanner unit (116), the plurality of moveable nozzles (114), the second scanner unit (118), and the alert generating unit (122). The central processing unit (120) is an integrated circuit and is envisaged to contain one or more CPUs (processor cores) along with memory and programmable input/output peripherals. The program memory in the form of ferroelectric RAM, NOR may flash, or OTP ROM is, included on the chip, along with a small amount of RAM.

The plurality of sensors (112), plurality of moveable nozzles (114), first scanner unit (116), second scanner unit (118), and an alert generating unit (122) are operationally connected with the central processing unit (120) through a communication network (not shown in figure). The communication network can be a short-range communication network and/or a long-range communication network, a wired or wireless communication network. The communication interface includes, but is not limited to, a serial communication interface, a parallel communication interface, or a combination thereof. The communication is established over maybe, but not limited to, wired network or wireless networks such as GSM, GPRS, CDMA, Bluetooth, Wi-Fi, ZigBee and intranet. Preferably, the one or more wireless communication modules are selected from one or more of a Wi-Fi, Bluetooth, and/or a Global System for Mobile (GSM) communication module.

In accordance with an embodiment of the present invention, the collection chamber (106) is operatively connected with the washing module (110). The collection chamber (106) is configured to collect the dirty water produced after hand wash. The outlet pipe (108) is attached to the collection chamber. The outlet pipe is configured to drain out dirty water.

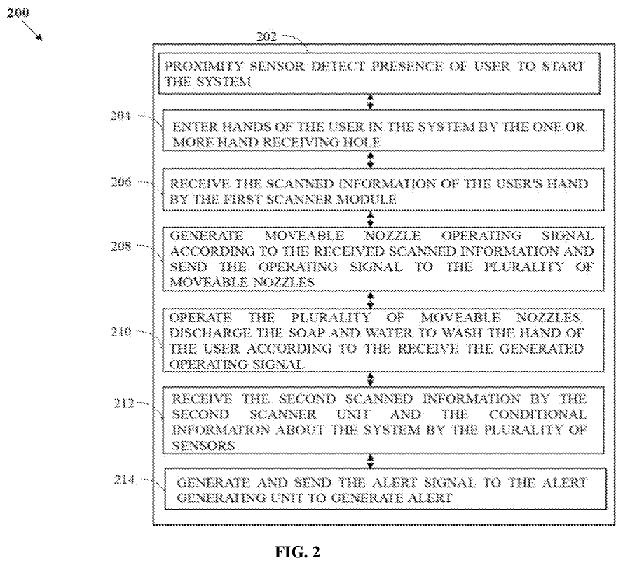

FIG. 2: illustrates an implementation of the artificial intelligence based smart hand sanitization system (100) for elderly, in accordance with an embodiment of the present invention. The method (200) of the artificial intelligence based smart hand sanitization system (100) for elderly involves detection (202) of the presence of the user by using the proximity sensor; enter (204) hands of the user in the system by the one or more hand receiving hole (124); receive (206) the scanned information of the user's hand by the first scanner unit (116); generate (208) a moveable nozzle operating signal according to the received scanned information and send the operating signal to the plurality of moveable nozzles (114); operate (210) the plurality of moveable nozzles (114), discharge the soap and water to wash the hand of the user according to the generated operating signal; receive (212) the second scanned information by the second scanner unit (118) and the conditional information about the system (100) by the plurality of sensors (114); generate (214) and send the alert signal to the alert generating unit (122) to generate the alert. The smart portable system (100) for hand wash analyzes the condition of the containers, hand after wash, and generate an alert to correct the issue accordingly.

In accordance with an embodiment of the present invention, the present invention is a detachable smart portable system (100) for hand wash which can be used by any institutes, hospital, and shopping mall. The user may be but is not limited to old, aged people (elderly), patients, small children.

While considerable emphasis has been placed herein on the specific features of the preferred embodiment, it will be appreciated that many additional features can be added and that many changes can be made in the preferred embodiment without departing from the principles of the disclosure. These and other changes in the preferred embodiment of the disclosure will be apparent to those skilled in the art from the disclosure herein, whereby it is to be distinctly understood that the foregoing descriptive matter is to be interpreted merely as illustrative of the disclosure and not as a limitation.

What is claimed is:

1. An artificial intelligence based smart hand sanitization system (100) for elderly comprising:
a. an inlet pipe (102) configured to fill liquids inside a system (100),
b. a liquid chamber (104) attached to the inlet pipe (102) having one or more separating walls to separately carry the received liquids;
c. a washing model (110) operationally connected with the liquid chamber (104), having one or more hand receiving holes (124) for washing hands, comprises:
i. a plurality of sensors (112) configured to detect conditional information about the system (100);
ii. a first scanner unit (116) configured to scan the hands of the user and generate a first scan information, wherein the one or more hand receiving holes (124) provide housing to a first scanner unit (116);
iii. a plurality of moveable nozzles (114) operationally connected with the first scanner unit (116);
iv. a second scanner unit (118) operatively connected with the plurality of moveable nozzles (114), configured to scan dirt and germs present in the hand of the user after wash and generate a second scan information;
v. an alert generating unit (122) operationally connected with the plurality of sensors (112), a second scanner unit (118), a liquid chamber (104), configured to generate an alert;
vi. a central processing unit (120) operationally connected with the first scanner unit (116), the plurality of moveable nozzles (114), the second scanner unit (118) and a alert generating unit (122), configured to
vii. receive the scanned information of the user's hand by the first scanner unit (116);
viii. generate moveable nozzle operating signal according to a received scanned information and send the operating signal to the plurality of moveable nozzles (114);
ix. operate the plurality of moveable nozzles (114), discharge the soap and water to wash the hand of the user according to the generated operating signal;
x. receive the second scanned information by the second scanner unit (118) and the conditional information about the system (100) by the plurality of sensors (112);
xi. generate and send the alert signal to the alert generating unit (122) to generate alert;
d. a collection chamber (106) operatively connected with washing module, configured to collect the dirty water produced after hand wash;
e. an outlet pipe (108) attached to a collection chamber configured to drain out dirty water.

2. The system (100) as claimed in claim 1, wherein the liquid is selected from a group comprising liquid soap, water, liquid antiseptic, sanitizer or combination thereof.

3. The system (100) as claimed in claim 1, wherein the conditional information about the system (100) are level of liquid, leakage of water from the collection chamber and the liquid chamber (104), presence of user near the system (100).

4. The system (100) as claimed in claim 1, wherein the plurality of sensors (112) is selected from a group comprising a water leakage sensor (ultrasonic sensor), a proximity sensor, hydrostatic pressure level sensor, radar level sensor, ambient light sensor, ultrasonic proximity, IR sensor proximity sensor, capacitive proximity sensor, passive IR sensor.

5. The system (100) as claimed in claim 1, wherein the first scan information is a type of wound, type of accessories, shape, and size of accessories, type of dirt, and germs.

6. The system (100) as claimed in claim 1, wherein the second scan information is whether the hands of user are properly cleaned or not, presence of dirt and germs after wash, presence of dirt in between fingers.

7. The system (100) as claimed in claim 1, wherein the first scanner unit (116) is selected from a group comprising an infrared laser scanner, an optical scanner, IR optical scanner, UV scanner, or a combination thereof.

8. The system (100) as claimed in claim 1, wherein the second scanner unit (118) is selected from a group comprising an infrared laser scanner, an optical scanner, IR optical scanner, UV scanner, or a combination thereof.

9. The system (100) as claimed in claim 1, wherein the alert generating unit (122) is selected from a group comprising a sound generator, flashing light, short message service, image-producing unit, or combination thereof.

10. The system (100) as claimed in claim 1, wherein the alert signal is about the issues related to the leakage of water from the collection chamber (106) and the liquid chamber (104), condition of filling level of liquid, not proper wash/or cleaning of hands, not proper working of the first scanner (116) and the second scanner (118), leakage of water through the inlet pipe (102) and outlet pipe (108).
